(19) United States
(12) Patent Application Publication
(10) Pub. No.: US 2005/0083484 A1
(43) Pub. Date: Apr. 21, 2005

(54) Ophthalmologic photographing apparatus

(76) Inventor: Kyoji Sekiguchi, Tochigi (JP)

Correspondence Address: MORGAN & FINNEGAN, L.L.P., 3 WORLD FINANCIAL CENTER, NEW YORK, NY, 10281-2101, US

(21) Appl. No.: 10/951,949

(22) Filed: Sep. 28, 2004

(30) Foreign Application Priority Data
Oct. 20, 2003 (JP) 2003-359743

(51) Int. Cl. A61B 3/14

(52) U.S. Cl. 351/206

(57) ABSTRACT

Images transcribed with an eye fundus portion and data card information from two images obtained by continuously photographing the data card and the eye fundus photographing are combined. A photographing light source is flashed, and an eye fundus image is obtained by a digital still camera. The photographed eye fundus image is converted into an electrical signal, and after that, it is stored in the memory of an image processing display portion. Subsequently, when a set data card is photographed, this photographed image is also converted into the electrical signal in the digital still camera, and after that, it is stored in the memory. When both of the photographing are completed, an eye fundus area is cut out from the eye fundus image, and a data area is cut out from the data card, and both of them are combined.

### BACKGROUND OF THE INVENTION

1. Field of the Invention

The present invention relates to an ophthalmologic photographing apparatus and a photographing method used for an ophthalmologic examination in an ophthalmologic hospital and the like.

2. Related Background Art

Heretofore, in general, an eye fundus camera, for example, similarly to Japanese Patent Application Laid-Open No. 2003-210407, has filled in an ID number, a name and other necessary items with a data card to identify an examinee, and transcribed them at the side of an eye fundus image at the time of fundus photographing.

FIG. 10 shows a configuration for such a transcription,
where an optical path switching mirror 2 and a photographing lens 3 are arranged in front of a imaging sensor 1 which photographs an eye fundus in a still image. In case of performing alignment for an eye to be examined, an optical path switching mirror 2 is arranged in the same way as a solid line, and at the time of photographing, the eye to be examined is photographed by a high sensitivity imaging sensor 4, and at the time of photographing the eye fundus, the optical path switching mirror 2 jumps up in the same way as a dotted line, and the eye fundus image is formed in the imaging sensor 1. At this time, a predetermined range of a data card C illuminated by an illumination lamp 5 is transcribed in the corner of the imaging sensor 1 through data card photographing optical systems 6, 7 and 8.

As evident from FIG. 10, to transcribe the data card C, it is necessary that the imaging sensor 1 is decentered from a photographing optical axis, and to transcribe the data card C, the arrangement of a complicated optical system and mechanical members is required, such as the illumination lamp 5 for illuminating the data card C by synchronizing with an eye fundus photographing and an electrical circuit for controlling the illumination lamp 5, data card photographing optical systems 6 to 8, and members and the like for shielding respective leaked beams from an eye fundus photographing optical system and the data card photographing optical systems 6 to 8.

While the data card C is required to be a size of about several centimeters since it is filled in by a person, if the size of a photographed image surface is 1/2 inch, an opposite corner will become 12.7 centimeters, which is small, and therefore, the photographing optical systems 6 to 8 require a compact optical system which is reduced one fourth to one fifth, and require compact optical members to be arranged in a narrow space.

Thus, to transcribe the eye fundus and the data card simultaneously on the same imaging sensor 1, a complicated mechanism and an optical system are required, and assembly and adjustment thereof take a lot of trouble, and the cost of achieving functions thereof becomes extremely expensive. Further, according to a conventional fundus camera, change of variable power and enlarged photographing change an imaging variable power, which causes the eye fundus to be transcribed across a whole imaging sensor, thereby eliminating a data portion in some cases.

### SUMMARY OF THE INVENTION

The present invention has been made in view of the above problems, and an object of the invention is to provide a suitable technology of an eye fundus image processing for displaying an eye fundus image outputted from an eye fudus camera used in ophthalmology and internal medicine.

In order to achieve the above object, for example, an ophthalmologic photographing apparatus of the present invention comprises the following arrangement.

That is, the ophthalmologic photographing apparatus comprises:
ophthalmologic imaging means for photographing an ophthalmologic image data, and a data card for displaying information associated with the ophthalmologic image data,
information photographing means for photographing the information by the data card, and
image storing means for storing the image information obtained by the ophthalmologic photographing means and the information photographing means,
wherein image combining means cuts out predetermined areas from the two pieces of the image information stored in the image storing means and combines them into one image.

In order to achieve the above object, for example, an ophthalmologic photographing method of the present invention comprises the following arrangement.

That is, the ophthalmologic photographing method comprises:
an ophthalmologic photographing step for photographing the ophthalmologic image data, a displaying step for displaying the information associated with the ophthalmologic image data by a data card,
an information photographing step for photographing the information by the data card, and
an image storing step for storing the image information obtained by the ophthalmologic photographing step and the information photographing step,
wherein the image combining step cuts out predetermined areas from the two pieces of the image information stored in the image storing means and combines them into one image.

Other features and advantages of the present invention will be apparent from the following description taken in conjunction with the accompanying drawings, in which like reference characters designate the same or similar parts through out the figure thereof.

### BRIEF DESCRIPTION OF THE DRAWINGS

The accompanying drawings, which are incorporated in and constitute a part of the specification, illustrate embodiments of the invention and, together with the description, server to explain the principles of the invention.

FIG. 1 is a block diagram of a first embodiment;

FIG. 2 is an explanatory drawing of a data card;

FIG. 3 is an explanatory drawing of an eye fundus photographing image;

FIG. 4 is an explanatory drawing of a data card photographing image;

FIG. 5 is an explanatory drawing of a combined image;

FIG. 6 is a timing chart;

FIG. 7 is a flowchart;

FIG. 8 is a block diagram of a data card transcription portion of a second embodiment;

FIG. 9 is an explanatory drawing of another data card; and

FIG. 10 is a block diagram of the data card transcription portion of a conventional eye fundus camera.

### DESCRIPTION OF THE PREFERRED EMBODIMENTS

Preferred embodiments of the present invention will now be described in detail in accordance with the accompanying drawings.

The present invention will be described in detail based on illustrated embodiments.

(First Embodiment)

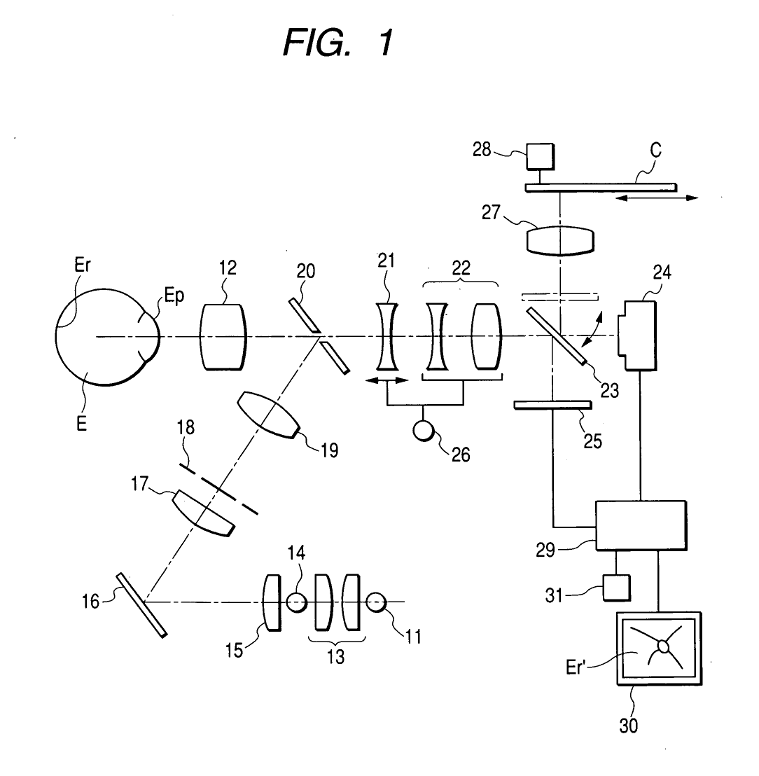

FIG. 1 shows a block diagram of a first embodiment of an eye fundus camera, where, on an optical path from an observation alignment light source 11 to an object lens 12, there are arrayed in order a condenser lens 13, a still image photographing light source 14 such as a strobe light and the like, a condenser lens 15, a mirror 16, a lens 17, an aperture 18 having a ring-shaped opening, a relay lens 19, and a perforated mirror 20 having an opening in the center.

On the optical path behind the perforated mirror 20, there are arrayed in order a focus lens 21, a photographing lens 22 having a variable power function, a switching mirror 23, and a high definition digital still camera 24 such as a digital camera, and in a reflecting direction of the switching mirror 23, there is arranged a moving image observation high sensitive camera 25. Further, the focus lens 21 and the photographing lens 22 are adjustable by a knob 26. In addition, on a reflecting side of the high sensitive camera 25 relative to the switching mirror 23, there is arranged a data card C through a lens 27, and a data card detection switch 28 is provided.

The output of the high sensitive camera 25 is connected to a monitor 30 for displaying an eye fundus image through an image processing display portion 29. Further, the image processing display portion 29 is connected to a display area enlarging portion 31, which can change a range to be displayed on the monitor 30 if required.

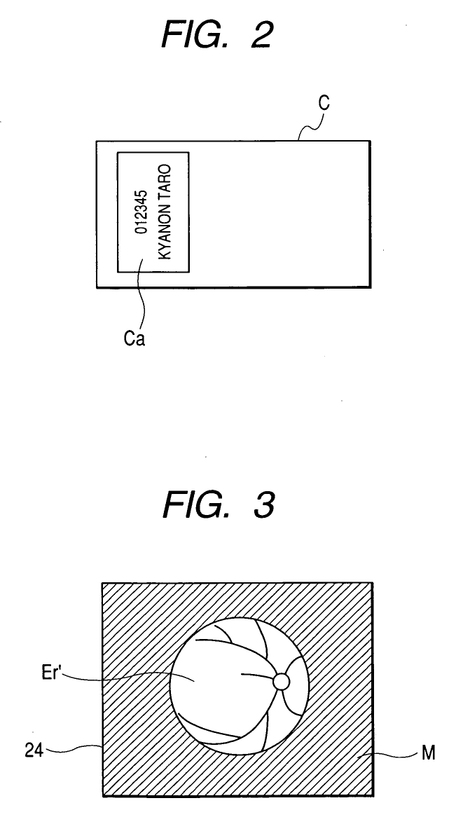

FIG. 2 shows the data card C, where a white board has a data area Ca distinguishably defined. In this area Ca, a number, a name and other pieces of information regarding a patient, which are required when interpretation of photograph is executed, are appropriately written by a pen and the like, and are erased when the photographing is over, and are used repeatedly.

In such a configuration, a light flux outgoing from the observation alignment light source 11 passes through the condenser lens 13, the still imaging light source 14, and the condenser lens 15, and is reflected upward by the mirror 16, and passes through the lens 17, the ring-shaped opening portion of the aperture 18, and the relay lens 19, and is reflected left and right by the perforated mirror 20, and passes through the object lens 12, and illuminates an eye fundus Er through the pupil Ep of an examinee's eye E.

The reflected light of the illuminated eye fundus image Er passes through a pupil Ep of the eye E to be examined, the object lens 12, the focus lens 21, and the photographing lens 22, and is reflected downward by the switching mirror 23 and is image-formed as an eye fundus image Er′ on the photographing image surface of the high sensitive camera 25. This eye fundus image Er′ is converted into an electrical signal, and is displayed on the monitor 30 through the image processing display portion 29.

While a radiophotograher observes this eye fundus image Er′, he confirms the state of a photographing area, an alignment, and a focus, and in case the focus deviates, by operating the focus knob 26, he performs a focus adjustment by moving the focus lens 21 associated with the movement of the focus knob 26 to the direction of an optical axis.

After completing adjustment of the focus, when an examiner depresses an unillustrated photographing switch, the switching mirror 23 evacuates the optical path, and the photographing light source 14 flashes. The light flux from the photographing light source 14 reaches the lens 15, and after that, passes through the same optical path as that of the light flux from the observation alignment light source 11, and illuminates the eye fundus Er.

The reflected light from the eye fundus Er thus illuminated passes from the pupil Ep through the object lens 12, a hole portion of the perforated mirror 20, the focus lens 21, and the photographing lens 22, and as shown in FIG. 3, is image-formed on the photographing image surface of the digital still camera 24, and the eye fundus image Er′ defined by the mask portion M is obtained. The photographed eye fundus image Er′ is converted into the electrical signal, and after that, it is stored in a still image memory within the image processing display portion 29.

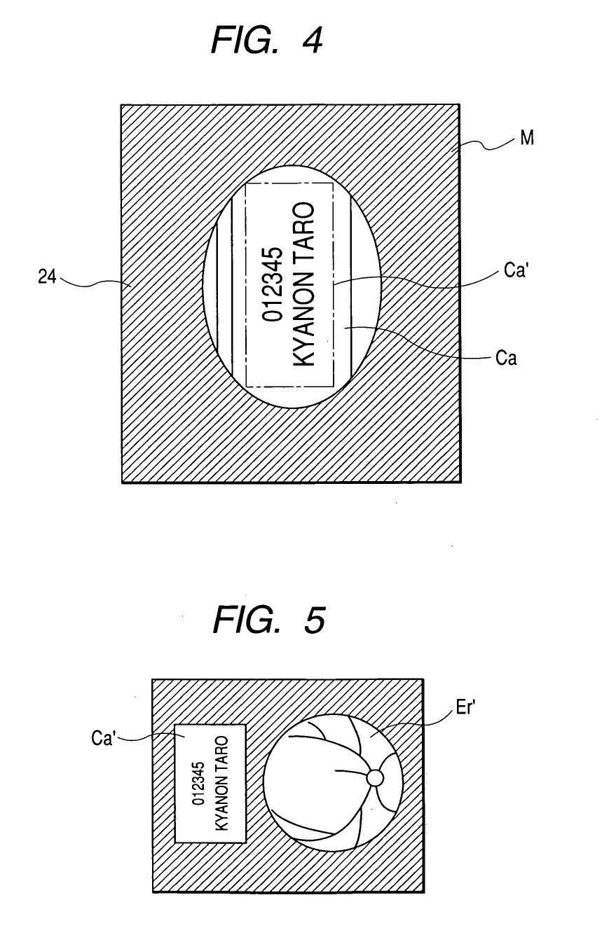

Subsequently, the switching mirror 23 is returned to the original position, and after that, the set data card C is photographed as shown in FIG. 4. The photographing of the data card C can take a relatively long time as the card C is fixed, and it is, therefore, possible to use a simple low light level light source such as a white color LED and the like. The photographing image surface of the data card C is also converted into the electrical signal in the digital still camera 24, and after that, it is stored in the still image memory of the image processing display portion 29.

When the photographing is completed, an eye fundus area is cut out from the image shown in FIG. 3, and a data area Ca′ shown by a broken line of the data card C shown in FIG. 4 is cut out. Since this data area Ca′ is fixed, its cutting out range becomes constant. The eye fundus changes its field angle when a variable power of photographing is changed, and though a range of photographing changes a little bit, since the mask portion M is a black level, it is easy to discriminate between a area of X direction and a area of Y direction. When respective images are cut out, respective sizes and positions thereof are calculated and arranged so that they match the image size of the digital still camera 24.

FIG. 5 shows a combined image, where the eye fundus image Er′ has its size adjusted and arranged such that the image above and below becomes the maximum in the right direction to meet the image size, and the size of the data area Ca′ is adjusted according to the size of a blank space at the left corner. At this time, the area cannot be reduced to the extent of being unable to read, and therefore, a limit on the reduction is regulated. When a degree of reduction for the data area Ca′ is below a regulated value, the size of the eye fundus Er′ is programmed such that it is reduced. This image is displayed in the monitor 30 and is stored in an unillustrated storage medium.

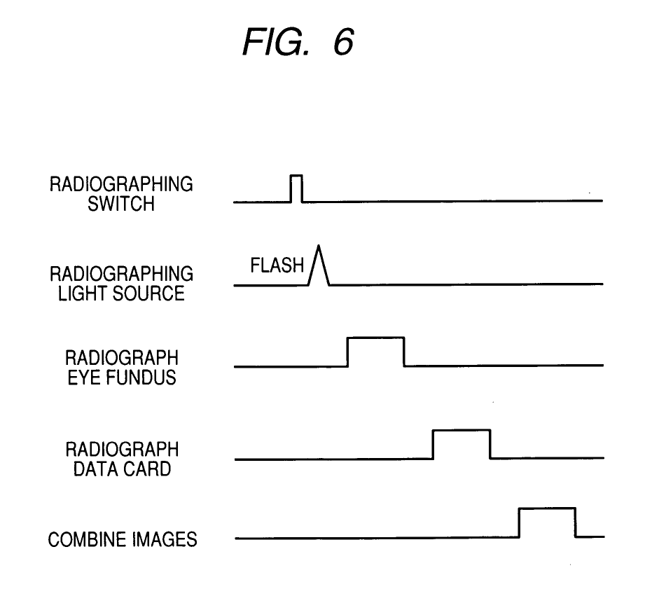

FIG. 6 is a timing chart at the time of photographing the eye fundus and the data card C. The abscissa axis is a time axis, from the upper axis of which are shown, respectively a photographing switch, a flash of the still imaging light source 14, and steps of photographing the eye fundus, photographing the data card C, and combining of images. First, when the alignment for the eye E to be examined and a setting of the data card C are completed and the photographing switch is depressed, the still image photographing light source 14 is flashed to illumine the eye fundus Er, and the eye fundus image Er′ is obtained by the digital still camera 24. Subsequently, the data card C is photographed, and a data card image is obtained. Predetermined areas are cut out from the two images, and the sizes thereof are adjusted and combined into one piece of an image. Thus shown the procedures in a sequential order.

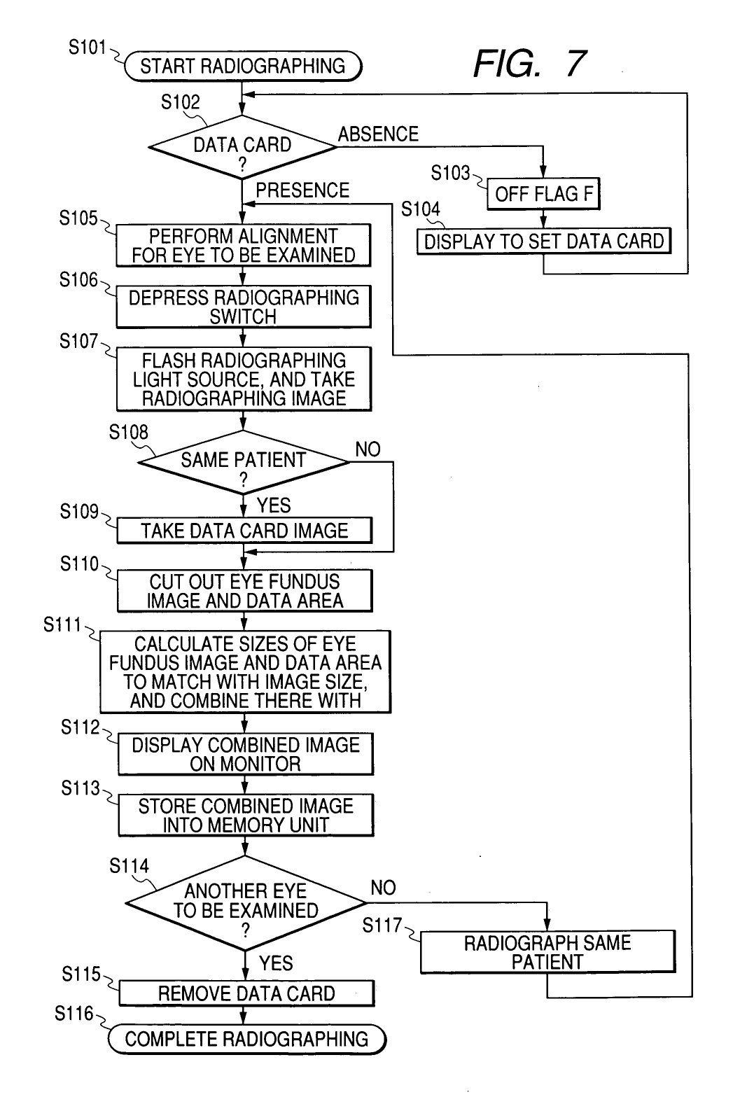

FIG. 7 shows a flowchart at the time of photographing a patient, where a photographing routine starts at step S101. The presence or absence of the data card C is checked at step S102. When the data card C is not set, a flag F is turned off at step S103. Then, [Please set the data card] is displayed at step S104, and the procedure returns to step S102. When information such as a patient number, a name and the like is filled in the data card C, and the setting of the card C is performed, the procedure advances to step S105 by the determination of step S102, and the display of [please set the data card] of step S104 disappears.

The align ment for the eye E to be examined is performed at step S105. The photographing switch is depressed at step S106, and the photographing light source 14 is flashed to obtain the eye fundus image Er′ at step S107. On/off of the flag F is determined at step S108. This determination is a determination also to see whether or not it is the same photographing of the examinee.

When the right and left eyes are photographed in succession, an exchange of data card C is not performed, and therefore, the photographing of the data card C is omitted on the second photographing. Even when it is the same eye to be examined, in case of reattempting to photograph it, there is no need to reattempt to photograph the data card C. When the flag F is turned off and the patient is different, the photographing of the data card C is performed at step S109, and when the flag F is turned on, the procedure advances to step S110 without photographing the data card C.

At step S110, the cutting out of an eye fundus image portion from the eye fundus image Er′ obtained at step S107 as well as the cutting out of the data area Ca′ of the data card C are performed. At step S111, the size of the eye fundus image Er′ and the size of the data area Ca′ are adjusted so as to match the image size of the digital still camera 24, thereby combining them into one piece of the image.

Since the heights of the eye fundus image Er′ and the data area Ca′ are the same as they are photographed by the same digital still camera 24, they are below the height of the combined image, and therefore, when the sum of the width of the eye fundus Er′ and the width of the data area Ca′ are below the width of the combined image, the gaps thus created are equally left and combined.

However, when (the width of the eye fundus Er′+the width of the data area Ca′) exceeds the width of the combined image, first, it is determined whether or not the data Ca′ falls within the value subtracting the width of the eye fundus Er′ from the combined image width at a reduction ratio within a predetermined magnification. When the data area Ca′ falls within the value, it is reduced by that reduction ratio and combined with the eye fundus image portion. When the area Ca′ does not fall within the value, first, it is reduced to the predetermined reduction ratio, and based on its image width, the width of the eye fundus Er′ is decided with the aspect ratio thereof made constant, and the eye fundus image portion is reduced, and after that, the area Ca′ is combined with the eye fundus image portion.

The combined image is displayed in the monitor 30 at step S112, and the combined image is stored at step S113. When the eye E to be examined is the same at step S114, the flag F is turned on, and the data card C is taken out at step S115, and the photographing is completed at step S116. When it is another eye to be examined at step S114, the procedure advances to step S117 and displays [the same patient is photographed] and the procedure returns to step S105.

(Second Embodiment)

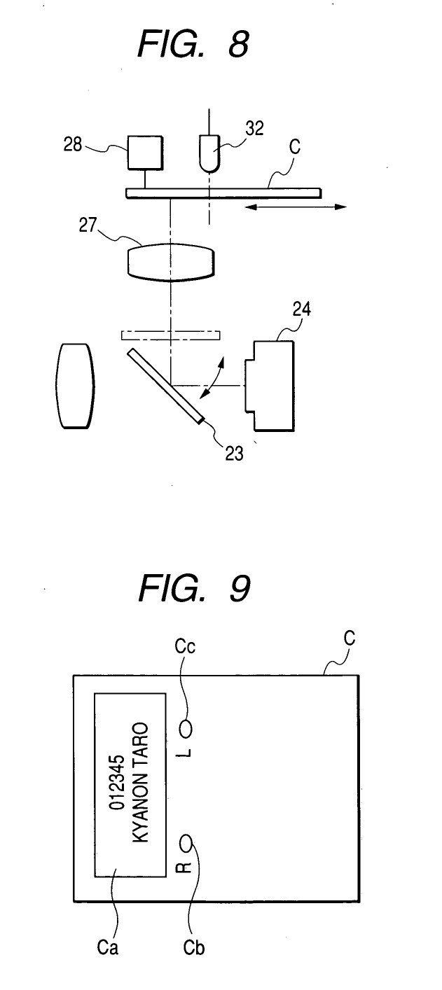

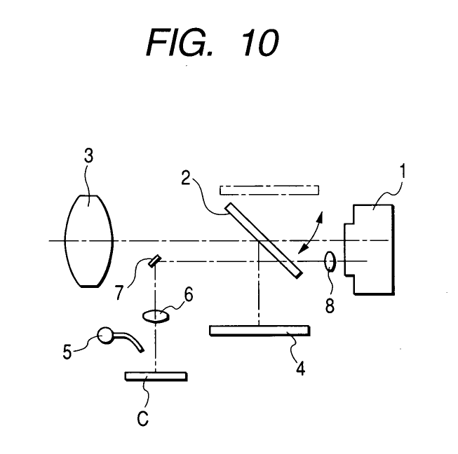

FIG. 8 shows a data card photographing portion of a second embodiment, wherein the outline of the configuration of another optical system is approximately the same as FIG. 1 except that two pieces of LED 32 are arranged above the fixed portion of a data card C.

As shown in FIG. 9, two opening portions Cb and Cc are perforated in a part within the photographing area of the data card C, and at the side of which are, for example, printed [R] and [L]. When the data card is inserted into the eye fundus camera, it is illumined from the back by the LED 32, and based on the detection result of the unillustrated left and right detection switches of the eye fundus camera, the state of the LED 32 being turned on or off is photographed, so that information on the data card C as well as information on the left and right eyes of the eye E to be examined where either of the openings Cb or Cc is photographed are combined together with the eye fundus image Er′.

In FIG. 8, while two openings Cb and Cc and two LEDs 32 for the left and right eyes are used, if the LED turns on by one opening and one LED, it may be for the right eye, and if the LED turns off, it may be for the left eye. Note that, when a transmission type material is used as the data card C, there is no need to provide the opening portions Cb and Cc, and it is enough to only illuminate characters R and L from the back thereof.

Further, it is possible to photograph and informationalize the photographing mode of the eye fundus camera, variable power information, and field angle information, which can be transcribed together with information on the eye fundus image Er′ and the data card.

Further, since the information on the examinee with respect to the type and the number of states is decided by the apparatus, these items are printed on the data card in advance, and are illuminated by the LED and the like according to the state, so that they can be transcribed.

A LED to display a time may be arranged in such a manner as to be photographed so that an elapsed time at the time of a timer photographing is photographed. Moreover, if the elapsed time at the time of photographing the eye fundus is held once and its value is displayed at the time of photographing the data card, it may match the elapsed time at the time of photographing the eye fundus image.

Further, instead of the data card C, a liquid crystal of a dot matrix type may be used in such a manner as to display handwritten input information in the liquid crystal from input means inputtable by a pen such as a tablet provided outside, and that information may be transcribed.

Since the image information is photographed by the difference of a luminance level and its coordinates are fixed by the position of the data card C, the positional detection thereof is electronically easy, and it is also easy to read these pieces of the information from the photographed image as an electronic data.

According to the ophthalmologic photographing apparatus and the ophthalmologic photographing method, the photographing of the eye fundus and the data card is continuously performed, and the processing of obtaining one combined image from the two obtained images is performed, and therefore, it is possible to output an ophthalmologic image in which the examinee's information is transcribed with a simple configuration and yet at a low cost without using a complicated optical system and mechanism.

Further, even in the ophthalmologic photographing apparatus of a configuration where the image size changes when a variable power photographing is performed, as described above, the size of the ophthalmologic image and the image information can be optimized and combined, and therefore, it is possible to output the ophthalmologic image in which the examinee's information is always transcribed.

Furthermore, the mode and state of the apparatus such as the left and right eyes information and the like can be easily transcribed, which are effective at the time of interpretation of the images, and the left and right information are extremely effective for the photographing of the fringes where both macula lutea and papillary area are not transcribed.

As described above, according to the present invention, the technology suitable for the ophthalmologic photographing apparatus and photographing method used for the ophthalmologic examination in the ophthalmologic hospital and the like can be provided.

It is only natural that the object of the present invention is achieved by providing to a system or a apparatus a recording medium (or storage medium) having recorded the program code of a software for realizing the function of the above described embodiment, and by reading and executing a program code stored in the recording medium by a computer (or CPU or MPU) of the system or the apparatus. In this case, the program code itself read from the recoding medium realizes the function of the above described embodiment, and the recording medium having recorded the program code configures the present invention.

Further, it is also natural that there are some cases included where, by executing the program code read by the computer, not only the function of the above described embodiment is realized, but also, based on the instruction of the program code, the operating system (OS) activated on the computer and the like performs a part or a whole of the actual processing, and by that processing, the function of the above described embodiment is realized.

Further, it is also natural that there are some cases included where the program code read from the recording medium is written in the feature expansion card inserted into the computer or the memory provided for the feature expansion unit connected to the computer, and after that, based on the instruction of the program code, the feature expansion card or the CPU and the like provided for the feature expansion unit performs a part or a whole of the actual processing, and by that processing, the function of the above described embodiment is realized.

In case the present invention is adapted to the above described recording medium, the program code corresponding to the flowchart described earlier is stored in that recording program.

As many apparently widely different embodiments of the present invention can be made without departing from the sprite and scope of thereof, it is to be understood that the invention is not limited to the specific embodiments thereof except as defined in the claims.

This application claims priority from Japanese Patent Application No. 2003-359743 filed on Oct. 20, 2003, which is hereby incorporated by reference herein.

1. A ophthalmologic photographing apparatus, comprising:
ophthalmologic photographing means for photographing an ophthalmologic image data;
a data card for displaying information associated with said ophthalmologic image data;
information photographing means for photographing information by said data card;
image storing means for storing image information obtained by said ophthalmologic photographing means and said information photographing means;
wherein image combining means cuts out a predetermined area from two pieces of said image information and image-combines it into one image.

2. The ophthalmologic photographing apparatus according to claim 1, wherein said data card can be filled in by handwriting.

3. The ophthalmologic photographing apparatus according to claim 1, wherein said data card can display handwritten information and the state information on said ophthalmologic photographing apparatus.

4. The ophthalmologic photographing apparatus according to claim 3, wherein the state information on the ophthalmologic photographing apparatus includes at leas one from among left and right eyes information, photographing mode information, variable power information, photographed field angle information, and information on a timer elapsed time.

5. The ophthalmologic photographing apparatus according to claim 1, wherein the photographing by said ophthalmologic photographing means and the photographing said information photographing means are continuously performed.

6. The ophthalmologic photographing apparatus according to claim 1, wherein said ophthalmologic photographing means and said information photographing means use common photographing means.

7. The ophthalmologic photographing apparatus according to claim 6, wherein an optical axis of said ophthalmologic photographing means and an photographing optical axis of said information photographing means are switchable by optical path switching means.

8. The ophthalmologic photographing apparatus according to claim 1, wherein said data card is made detachably insertable, and at the time of continuous photographing in an inserted state, said data card is not photographed.

9. An ophthalmologic photographing method, wherein, when an ophthalmologic image photographed by ophthalmologic photographing means as well as image information obtained by photographing the data card which displays information associated with said ophthalmologic image are stored, and a predetermined area is cut out from said stored ophthalmologic image and image information, and is image-formed into one image, the size of the predetermined area image cut out from said ophthalmologic image is preferentially decided, and after that, the size of a predetermined area image cut out from said information image is decided, thereby performing a combined processing.

10. An ophthalmologic photographing method, comprising:
an ophthalmologic photographing step for photographing an ophthalmologic image data;
a displaying step for displaying information associated with said ophthalmologic image data by a data card;
an information photographing step for photographing information by said data card;
an image storing step for storing image information obtained by said ophthalmologic photographing step and said information photographing step;
wherein an image combining step cuts out a predetermined area from two pieces of said image information stored in the image storing step and image-combines it into one image.

11. A program for allowing a computer to execute a processing step of an eye fundus image processing method according to claim 10.
